(19) United States
(12) Patent Application Publication
(10) Pub. No.: US 2006/0149035 A1
(43) Pub. Date: Jul. 6, 2006

(54) Method for the synthesis and selective biocatalytic modification of peptides, peptide mimetics and proteins

(76) Inventors: Rainer Rudolph, Halle/Saale (DE); Frank Bordusa, Rossbach (DE); Nicole Wehofsky, Leipzig (DE)

Correspondence Address: Roche Diagnostics Corporation, Inc., 9115 Hague Road, PO Box 50457, Indianapolis, IN, 46250-0457, US

(21) Appl. No.: 10/526,163

(22) PCT Filed: Sep. 1, 2003
(86) PCT No.: PCT/EP03/09694

§ 371 Date: Feb. 21, 2006

(30) Foreign Application Priority Data
Aug. 30, 2002 (DE) 102 40 098.9

(51) Int. Cl. C07K 1/02

(52) U.S. Cl. 530/333

(57) ABSTRACT

The present invention relates to a method for the synthesis of peptides, peptide mimetics and/or proteins and/or for the selective N-terminal modification of peptides, peptide mimetics and/or proteins, with the steps of: a) providing an amino component, said amino component having at least one amino acid, b) providing a carboxyl component, said carboxyl component having a leaving group on the carboxyl group, and said carboxyl component being a compound having at least one amino acid or a compound having at least one label or reporter group, c) reacting said amino component and said carboxyl component in a reaction medium which has one or more ionic liquids, in the presence of a protease, peptidase and/or hydrolase, to form a peptide bond between the amino component and the carboxyl component with elimination of the leaving group.

The present invention relates to a method for the enzymatic synthesis and/or selective modification of peptides, peptide mimetics and/or proteins using ionic liquids, and to the use of ionic liquids as an exclusive reaction medium or in combination with water and/or organic solvents for the suppression of hydrolytic and proteolytic side reactions.

The synthesis and selective modification of peptides, peptide mimetics and proteins has increasing significance for the systematic study of structure-functional relationships of polypeptides as functional gene products and makes a crucial contribution to the discovery of novel effective therapeutics (cf. H.-D. Jakubke, Peptide: Chemie und Biologie, Spektrum Akademischer Verlag, Heidelberg, Berlin, Oxford, 1996). However, a significant problem in their synthesis or selective modification is the lack of selectivity and universality of chemical methods, and the substrate limitation or the occurrence of numerous side reactions in the case of the use of enzymes as catalysts.

In principle, the chemical methods developed in peptide chemistry can be used for the synthesis of peptides, peptide mimetics and proteins. However, these are subject to considerable limitations with increasing complexity of the products. While peptides having an average chain length of 50-60 amino acids are obtainable directly by solid-phase peptide synthesis, a further chain extension leads, owing to the coupling yields which are not quantitative in every case, frequently to the accumulation of a multitude of by-products, which both lead to a reduction in the synthesis yields and complicate or prevent the purification of the desired product. Current methods for the synthesis of relatively long polypeptides or of proteins are therefore based on the condensation of synthetically prepared peptide fragments, even though the connection of fully protected peptide fragments is possible only in exceptional cases owing to the frequently very low solubility of the reactants.

The methods, developed on the basis of the concept, first proposed in 1953, of the molecular bracket for chemical CN ligations of unprotected peptide fragments (T. Wieland et al., Annalen 1953, 583, 129; M. Brenner et al., Helv. Chim. Acta 1957, 40, 1497), of amine and thiol capture (D. S. Kemp et al., J. Org. Chem. 1975, 40, 3465; N. Fotouhi et al., J. Org. Chem. 1989, 54, 2803), of natural chemical ligation (M. Schnölzer, S. B. H. Kent, Science 1992, 256, 221; P. E. Dawson et al., Science 1994, 266, 776) or else of the aldehyde method (C.-F. Liu, J. P. Tam, Proc. Natl. Acad. Sci. USA 1994, 91, 6584) do proceed selectively, but require for their realization quite specific N- or C-terminal amino acid residues, so that their applicability is subject to sequence-specific prerequisites. In the case of the currently favored native chemical ligation, a synthetic peptide is connected using a C-terminal thioester moiety to a second peptide or protein which has to contain an N-terminal cysteine residue. Utilizing knowledge of protein splicing, native chemical ligation has been further developed to an intein-mediated protein ligation (expressed protein ligation, EPL; cf., inter alia, T. W. Muir et al., Proc. Natl. Acad. Sci, USA 1998, 95, 6705; G. J. Cotton et al., J. Am. Chem. Soc. 1999, 121, 1100), in which the thioester moiety of the carboxyl component from a recombinant protein which has been fused with a cleavage-competent intein and is formed by thiolytic cleavage. In addition to the need for a cysteine residue at the N-terminus of the amino component, a further general disadvantage lies in the partial epimerization of the C-terminal amino acid residue which cannot be ruled out, since the thioester which forms (at least when thiophenol is used as a catalyst) can be attacked nucleophilically not only after the transesterification but also directly by the terminal α-amino group of the added amino component.

Catalytic synthesis methods offer the advantage of higher flexibility with regard to the peptide bond to be synthesized, although no universal peptide ligase with preparative relevance is yet known, at least from nature. For instance, catalytic antibodies (cf., inter alia, P. G. Schultz, R. A. Lerner, Science 1995, 269, 1835; G. MacBeath, D. Hilvert, Chem. Biol. 1996, 3, 433; D. B. Smithrub et al., J. Am. Chem. Soc. 1997, 119, 278) exhibit CN ligase activity, as do synthetic peptide ligases based on a coiled-coil motif of GCN4 (K. Severin et al., Nature 1997, 389, 706) or on a peptide template consisting of a strongly acidic coiled-coil peptide (S. Yao, J. Chmielewski, Biopolymers 1999, 51, 370). All of these cases are without doubt interesting starting points for the design of peptide ligases, but they entail specific prerequisites for ligations and their general applicability is consequently very greatly limited. Although the utilization of the reverse catalysis potential of peptidases (cf., inter alia, W. Kullmann, Enzymatic Peptide Synthesis, CRC Press, Boca Raton, 1987; H.-D. Jakubke, Enzymatic Peptide Synthesis, in: The Peptides: Analysis, Synthesis, Biology, Vol. 9, (Eds.: S. Udenfriend, J. Meienhofer), Academic Press, New York, 1987, Chapter 3) offers the possibility in principle of enzymatically connecting peptide segments under specific prerequisites, neither is the irreversibility of the connected specific peptide bond guaranteed nor can undesired proteolytic cleavages in the segments to be connected or in the end product be ruled out α priori when potential cleavage sites for the peptidase used are present there. Although reengineering of various peptidases, for example subtilisin, improves the catalysis potential for peptide bond formation and have also been demonstrable by demanding fragment condensations (cf., inter alia, D. Y. Jackson et al., Science 1994, 266, 243), it is not possible in this way to eliminate the disadvantages outlined above. Although the substrate mimetics concept developed for CN ligations of peptide and protein segments (F. Bordusa et al., Angew. Chem. 1997, 109, 2583; Review: F. Bordusa, Braz. J. Med. Biol. Res. 2000, 72, 469) has the advantage of irreversibility, it likewise requires the use of synthetic, proteolytically inactive protease variants in order to prevent competitive cleavages within the biopolymers to be connected.

For the modification of peptides, peptide mimetics and proteins, chemical processes (cf. T. Imoto, H. Yamada, Chemical Modification, in Protein Function. A Practical Approach (T. E. Creighton, ed.) pp. 247-277, IRL Press, 1989; G. E. Means, R. E. Feeney, Chemical Modification of Proteins, Holden-Day, 1971) played, and still play, a significant role in protein research. Despite the rapid progress of NMR technology which in the last decade has enabled full signal assignment and thus elucidation of the 3D structure of proteins up to 150-200 amino acids (without modification), chemical modification is still also a tool for 3-dimensional structure determination in solution, since large proteins are not amenable to NMR structural analysis and X-ray structural analysis requires protein crystals which cannot be obtained in very many cases.

Since N-terminal α-amino groups are preferred targets of selective modifications, the ε-amino groups of lysine radicals occurring ubiquitously in proteins and peptides do not allow any targeted introduction of label and reporter groups at the N-terminus. Chemical acylation reactions are carried out with anhydrides or primarily with active esters, for example N-hydroxysuccinimide or 4-nitrophenyl esters, with which, however, other side chain functions of proteinogenic amino acid residues might also react and thus rule out selective N^{α} modification. Only the phenylacetyl radical has been introduced enzymatically as a protecting group for amino acids in the context of peptide syntheses with determination of specificity by penicillin acylase in the reverse of the native action (R. Didziapetris et al., FEBS Lett. 1991, 287, 31) and cleaved off again by the same enzyme (cf. Review: A. Reidel, H. Waldmann, J. prakt. Chem. 1993, 335, 109). Apart from this direct protecting group introduction, the only methods described have been those which are based on a transfer of already N-terminally labeled amino acid or peptide derivatives with peptidase-specific amino acid residues in the P₁ position under the catalysis of peptidases and inevitably do not have irreversibility. An exception thereto is the substrate mimetics concept originally developed for CN ligations of peptide and protein segments (F. Bordusa et al., Angew. Chem. 1997, 109, 2583: Review: F. Bordusa, Braz. J. Med. Biol. Res. 2000, 72, 469). Although this methodology has the advantage of directive and selective introduction of label and reporter groups, it requires, as already mentioned, the use of synthetic, proteolytically inactive protease variants as biocatalysts in order to prevent competitive cleavages of the biopolymers to be labeled.

Hydrolytic and proteolytic side reactions of hydrolases used for the synthesis and modification of peptides, peptide mimetics and proteins can be suppressed not only by targeted enzyme engineering but also by manipulations of the reaction medium. The literature describes the use of monophasic mixtures of water and organic solvents, of analogous biphasic systems in the case of immiscibility of water and organic solvent, of pure organic solvents with virtually no or only a very small water content, of frozen or supercooled aqueous or organic systems, of supercritical solutions and of heterogeneous eutectic mixtures with virtually no or only a very small solvent content (cf., inter alia, W. Kullmann, Enzymatic Peptide Synthesis, CRC Press, Boca Raton, 1987; H.-D. Jakubke, Enzymatic Peptide Synthesis, in: The Peptides: Analysis, Synthesis, Biology, Vol. 9, (Eds.: S. Udenfriend, J. Meienhofer), Academic Press, New York, 1989, Chapter 3). However, virtually all of these methods bring about an often dramatic reduction in the enzyme activity or stability and in some cases require considerable apparatus complexity. An additional factor is that only a few have been investigated at all with regard to their usability for the synthesis and modification of relatively long-chain biopolymers. However, even in such cases, none of these methods has hitherto been able to demonstrate the efficiency and universality required for routine application.

A novel class of solvents is represented by salts which have a low melting point. For these solvent systems also referred to as ionic liquids, a stabilizing influence on proteins and enzymes was demonstrated in initial studies (Review: C. M. Gordon, Appl. Catal. A: Gen. 2001, 222, 101). Simple model reactions with lipases and galactosidases have additionally shown that these liquids have a positive effect on the reaction rate and sometimes even on the selectivity of the enzymatic reactions (U. Kragl et al., Chimica Oggi 2001, 19, 22; T. L. Husum et al., Biocatal. Biotrans. 2001, 19, 331; S. H. Schofer et al., Chem. Commun. 2001, 425). The example of a simple amino acid ester substrate has additionally demonstrated that the serine proteases chymotrypsin and subtilisin too are enzymatically active in reaction systems having a high proportion of such liquids and catalyze both the hydrolysis of the ester and the transesterification thereof (J. A. Laszlo, D. L. Compton, Biotechnol. Bioeng. 2001, 75, 181; T. L. Husum et al., Biocatal. Biotrans. 2001, 19, 331). With reference to the synthesis of Z-Asp-Phe-OMe from Z-Asp-OH and H-Phe-OMe by the metalloprotease thermolysine, the suitability in principle of ionic liquids for the protease-catalyzed connection of two amino acids under equilibrium-controlled synthesis conditions has additionally been demonstrated (M. Erbeldinger et al., Biotechnol. Prog. 2000, 16, 1131). It is, though, completely unknown whether peptide fragments can be connected selectively by proteases in a kinetically controlled reaction in such liquids and whether a selective introduction of reporter and label moieties on the N-terminus of peptides and proteins is catalyzed by proteases under these conditions. In the same way, it is unclear what influence ionic liquids have on the extent of proteolytic side reactions on the reactants and hydrolytic side reactions on the ester substrate used.

It is an object of the present invention to provide a method for the enzymatic synthesis and modification of peptides, peptide mimetics and proteins, which overcomes the disadvantages of the methods described in the prior art. It is a further object of the present invention to provide a process in which a sequence-independent synthesis, in particular ligation and N-terminal modification, is effected regio- and stereoselectively without proteolytic and hydrolytic side reactions on the reactants or the reaction products.

According to the invention, the object is achieved by a method for the synthesis of peptides, peptide mimetics and/or proteins and/or for the selective N-terminal modification of peptides, peptide mimetics and/or proteins, with the steps of:
a) providing an amino component, said amino component having at least one amino acid,
b) providing a carboxyl component, said carboxyl component having a leaving group on the carboxyl group, and said carboxyl component being a compound having at least one amino acid or a compound having at least one label or reporter group,
c) reacting said amino component and said carboxyl component in a reaction medium which has one or more ionic liquids, in the presence of a protease, peptidase and/or hydrolase, to form a peptide bond between the amino component and the carboxyl component with elimination of the leaving group.

A preferred embodiment provides that the method according to the invention further comprises the step of:
d) isolating or enriching the resulting peptide, peptide mimetic and/or protein by methods known per se.

The present invention further relates to the use of ionic liquids as an exclusive solvent or in combination with water and/or organic solvents for the synthesis and/or N-terminal modification of peptides, peptide mimetics and/or proteins. The present invention also relates to the use of a protease, peptidase and/or hydrolase for the synthesis and/or N-terminal modification of peptides, peptide mimetics and proteins, said peptide, peptide mimetic and protein or N-terminally labeled species thereof being prepared by ligation of an amino component and a carboxyl component, and said carboxyl component having a leaving group.

Further embodiments are evident from the subclaims and the description which follows.

According to the invention, peptides refer to condensation products of amino acids having about 2-10 amino acids. According to the invention, polypeptides refer to condensation products of amino acids having about 10-100 amino acids and, according to the invention, the term protein is used for condensation products of amino acids which have more than about 100 amino acids, the literature regarding the transition between the two terms as being fluid.

According to the invention, peptide mimetics refer to compounds which imitate or antagonize the biological activity of a peptide without themselves having a classical peptide structure composed exclusively of coded amino acids. Examples of peptide mimetics are not only entirely nonpeptide organic compounds (for example the morphine or naloxone composed of cycloaliphatic and aromatic structures) but also those which have modified amino acids (e.g. N, α- and β-alkylated amino acids; C_{α}-C_{β} and N—C_{β} cyclized amino acids; peptides with modified side chains, e.g. α-,β-dehydrogenated amino acids, nitrotyrosine, etc.), and also cyclic peptide analogs (cyclization of N-terminus with C-terminus or amino acid side chain; cyclization of C-terminus with amino acid side chain or cyclization of amino acid side chains with amino acid side chain) and peptides with modified peptide bonds, for example thioamides, ketomethylenes, ethylenes, methylenamines or else retro-inverso derivatives, and the like. Retro-inverso derivatives are compounds having the peptide backbone structure R—C—NH—CO—C—R′ in which the position of the amino function and of the carboxylic acid function are exchanged in comparison to normal peptide bonds, while normal peptide bonds have the structure R—C—CO—NH—C—R′.

In contrast to salt melts, ionic liquids are salts which melt at low temperatures (<100° C.) and consist exclusively of ions (Lit.: see, for example, T. Welton, Chem. Rev. 1999, 2071-2083). By this definition, water is thus not ionic liquid. Characteristic features are their low symmetry, low intermolecular interactions and good charge distribution. Typical cations contain quaternized heteroatoms, for instance quaternized ammonium or quaternized phosphonium ions. Subgroups are, for example, N-alkylated imidazolium ions such as 1-ethyl-3-methylimidazolium, 1-butyl-3-methylimidazolium; N-alkylated pyridinium ions such as 4-methyl-N-butylpyridinium or analogously substituted ammonium and phosphonium ions. Typical anions may be either of inorganic or organic nature, for example chloride, bromide, chloroaluminate, nitrate, benzenesulfonate, triflate, tosylate or else tetrafluoroborate.

Starting from the phenomena observed previously in the prior art that the full, but also partial, replacement of water as the reaction medium by a reaction-inert solvent leads to an activity loss up to inactivity of the enzyme, the present invention is based on the surprising discovery that it is possible to connect a carboxyl component provided with a leaving group, the carboxyl component being a peptide, peptide mimetic, protein or a label or reporter moiety, under enzyme catalysis at high synthesis rate and selectivity, using ionic liquids as an exclusive solvent or in combination with water and/or organic solvents as a reaction medium, with an amino component which is preferably a peptide, peptide mimetic and protein.

It was also surprising in this context that the side reactions which typically proceed, such as the hydrolysis of the bond between carboxyl component and leaving group, and also the proteolysis of the peptide bonds corresponding to the specificity of the enzyme, in the reactants or the products of the reaction virtually do not proceed. The carboxyl component is provided with the leaving group typically by connecting the leaving group in ester, thioester or amide form to the C-terminal carboxyl function of the carboxyl component. The method according to the invention is thus based on the surprising discovery that ionic liquids or mixtures thereof, in contrast to virtually all other organic solvents, have a favorable influence on the synthesis activity of the enzyme and simultaneously virtually fully suppress the undesired side reactions which are typically mediated by the water solvent. The combined use of ionic solvents with carboxyl components which contain enzyme-specific leaving groups also allows independence of the synthesis activity from the original substrate specificity of the enzyme to be achieved, which crucially increases the scope of synthetic application of the method.

It has been found that all serine and cysteine proteases investigated so far exhibit the above-described behavior and are thus particularly suitable in the context of the method according to the invention for the synthesis and modification of peptides, peptide mimetics and proteins. Further suitable enzymes may be obtained in the context of screening processes using suitable model reactions, as can be carried out on the basis of the technical teaching disclosed herein.

In the context of such a screening process, the procedure is to investigate the synthesis activity of proteases, peptidases and/or hydrolases in ionic liquids or mixtures thereof by means of synthetic model reactions. To this end, in the simplest case, a carboxyl component consisting of one amino acid (conventional carboxyl component or substrate mimetic) is incubated with an amino component, in the simplest case one amide or amino acid, but preferably a peptide, and the protease, peptidase or hydrolase to be tested. The tolerance of ionic liquids by the enzyme is indicated by product formation in the course of the subsequent incubation phase. The product formation itself may be analyzed, for example, by means of HPLC or other chromatographic separation methods.

According to the invention, preferred proteases are cysteine proteases or serine proteases. However, it is possible in principle to use all other known types of proteases, i.e. aspartate proteases or metalloproteases too. Useful further hydrolase groups are in particular lipases or esterases. According to the invention, the peptidases (EC 3.4.11-3.4.19) used may in principle be the known peptidase subgroups. For the definition of hydrolases, peptidases and proteinases, reference is made in particular to Römpp Chemielexikon, 9th edition, 1989-1992.

A further advantage of the method according to the invention is based on the regiospecificity of the enzymes used and the absent risk of racemization compared to most chemical processes. This is advantageous insofar as reactants having chiral centers and other acylatable functions can be used without experimentally complex temporary and selective blocking measures which lead to additional side reactions. The only exceptions are the introduction, necessary in some cases, of N-terminal protecting groups into the carboxyl component, which becomes necessary especially when the N-terminal sequence of the carboxyl component has a higher specificity for the enzyme than the N-terminal sequence of the amino component. In addition, in contrast to the selective chemical methods, virtually no restrictions exist with regard to the sequence of the reactants entering into reaction.

According to the invention, the enzymes used may be proteases, peptidases and/or hydrolases. These preferably have a selectivity or specificity for the leaving group and/or certain amino acids or amino acid ranges of the carboxyl component. According to the invention, the leaving group and/or these amino acids or amino acid ranges may preferably be compounds naturally recognized by the enzyme used. According to the invention, they may preferably also be structurally similar compounds (substrate mimetics).

The terms amino component and carboxyl component, as used herein, are defined relatively to the polypeptide to be synthesized. The term amino component refers to a chemical compound which provides at least one amino group which reacts with a carboxyl group or a derivative thereof, for example a carboxyl group which has been derivatized with a leaving group, to form a peptide bond. The amino component is preferably an amino acid, more preferably a polypeptide or protein. In the latter case, the polypeptide or protein has both an amino end and a carboxyl end. The carboxyl end is either in unprotected or protected form. To avoid a reaction with another reactive group, for example the amino group or a further molecule of the amino component, the carboxyl group of the amino component is typically and preferably in nonactivated form. It is further preferred that the N^{α}-amino function of the amino component is present unprotected, and this N^{α}-amino function reacts with the carboxyl function of the carboxyl component.

The carboxyl component is preferably a label or reporter group provided with a carboxyl function, or is an amino acid, or, more preferably, is a polypeptide or protein. The carboxyl function or group of the carboxyl component which reacts with the amino group, generally the N-terminal amino group, of the amino component to form a peptide bond is typically and preferably activated.

Preference is given in accordance with the invention to the leaving group of the carboxyl component being selected from the group consisting of unsubstituted and substituted —O-alkyl-, —O-aryl-, —S-alkyl-, —S-aryl radicals, —NH-alkyl-, —NH-aryl-, —N,N-dialkyl-, —N,N-diaryl- and —N-aryl-N-alkyl-radicals, and preference is likewise given to the leaving group of the carboxyl component being substituted by one or more carboxylic acid radicals, sulfonic acid radicals or sulfonates. In this enumeration, the term alkyl also embraces cycloalkyl and heterocycloalkyl. Useful heteroatoms are in particular N, O and S. Preference is given to cycloalkanes or heterocycloalkanes having 5-6 ring carbon atoms. According to the invention, the alkyl radicals include n-alkyl radicals and branched alkyl radicals, and preference is given among these to n-alkyl radicals having 1-5 carbon atoms.

In this enumeration, the term aryl embraces in particular substituted and unsubstituted phenyl which is preferably substituted on the phenyl ring by guanidino and/or amidino. The term aryl here also embraces fused ring systems, preferably biphenyl, and nonfused ring systems such as naphthyl, which may in turn preferably be substituted by guanidino and/or amidino groups, and heteroanalogous systems such as quinoline or isoquinoline. The term aryl here also embraces heteroaromatic compounds having 5-6 ring atoms, in which one or more ring carbon atoms are preferably replaced by N, O and/or S. Examples are pyridine, thiophene, furan, pyrazole or imidazole radicals.

Particular preference is given to the leaving group of the carboxyl component being a 4-guanidinophenyl, 4-amidinophenyl, 4-guanidinophenylthio or 4-amidinophenylthio radical, or a compound structurally homologous thereto.

In the carboxyl component, the leaving group preferably forms an ester or an amide with the carboxyl group of the carboxyl component, more preferably at the C-terminal carboxyl group of the carboxyl component. As already detailed, specific recognition of the leaving group ultimately modifies the enzymatic activity of the enzyme to the effect that peptide fragments or label and reporter groups are also connected by the protease, hydrolase or peptidase instead of reactants having enzyme-specific amino acid residues.

For arginine-specific proteases, for example trypsin, such a specificity-mediating action was detected for the 4-guanidinophenyl ester leaving group. An analogous function can also be observed for amidinophenyl esters. In addition, owing to the structural homology, the 4-guanidinophenyl thioester and 4-amidinophenyl thioester analogs have a similar action and additionally have advantages in chemical synthesis. Structural homologs of these compounds are likewise useful as specificity-mediating leaving groups.

Structurally homologous compounds are derivatives of these compounds with a basic moiety, for example amino, amidino, guanidino and imino moieties, which interact with specificity-determining amino acid residues of the protease, i.e. especially those amino acid residues which interact directly or indirectly with the substrate, or those which influence the catalytic reaction, and which have an aliphatic or aromatic basic structure with, for example, a chain length between one and six methylene units, or benzene, naphthalene or indole basic structures, between the specific basic moiety and the ester or amide function as a connecting element between the carboxyl group of the carboxyl component and the leaving group. In the figurative sense, this likewise applies to enzymes having a primary specificity for glutamic acid or aspartic acid, for example V8 protease, with the difference that, instead of the basic moieties on the aliphatic or aromatic basic structures, an acidic moiety such as carboxylic and sulfonic acid groups are bonded. In an analogous manner, esters or amides which consist only of the basic structures mentioned, i.e. do not have any basic or acidic groups, constitute carboxyl components for enzymes having a preferential specificity for hydrophobic amino acid residues, for example chymotrypsin and subtilisin.

According to the invention, the leaving group is preferably adjusted to the specificity of the protease, peptidase and/or hydrolase used.

In a further preferred embodiment, the carboxyl component containing the leaving group has the following structure:
Y—(Xaa)ₙ-R
where Y=an N-terminal protecting group or is H,
   Xaa=any (α-amino acid, β-amino acid or a derivative thereof, or is a label or reporter group,

R is a leaving group, in particular a leaving group which is selected from the group consisting of unsubstituted and substituted —O-alkyl, —O-aryl-, —S-alkyl-, —S-aryl-radicals, preferably 4-guanidinophenyl, 4-amidinophenyl, 4-guanidinophenylthio-, 4-amidinophenylthio radicals, each of which may be substituted by sulfonic acid groups or sulfonates, and also structural homologs thereof,

n is an integer of from 1 to 1000, preferably from 30 to 500, more preferably from 30 to 250.

In this context, the terms alkyl and aryl are as defined above and also embrace as above cycloalkanes, heterocycloalkanes, fused ring systems and nonfused ring systems, and also the abovementioned preferred embodiments.

It is likewise preferred that the carboxyl component is a label or reporter group selected from fluorescent labels containing carboxyl groups, such as fluorescein, rhodamine, tetramethylrhodamine, 2-aminobenzoic acid; isotopic labels containing carboxyl groups, such as ¹³C—, ¹⁵N— and ¹⁷O-containing amino acids or peptide fragments; spin labels such as nitroxide label-containing amino acid and fatty acid derivatives; biotin; crosslinking agents containing carboxyl groups, such as diazoacetate, diazopyruvate, p-nitrophenyl-3-diazopyruvate; 2-( 1,2-dithiolan-3-yl)acetate; N,N′-1,2-phenylenedimaleimide; N,N′-1,4-phenylenedimaleimide. All derivatives mentioned have a carboxyl group which has been provided with one of the above-defined leaving groups before the enzymatic reaction. In this respect, the fluorescent labels mentioned are not the complete carboxyl component, but rather only the part which is transferred to the amino component.

The connection of the amino component and the carboxyl component forms a polypeptide or selectively modified polypeptide or analog thereof, the C-terminal end of the amino component corresponding to the C-terminal end of the polypeptide and the amino-terminal end of the carboxyl component corresponding to the amino end of the polypeptide ligated under the influence of the enzyme or to the label and reporter group introduced. The length of the synthesized or modified polypeptide is at least two amino acids. Typically, the length of the polypeptide or protein prepared in accordance with the invention will have a size of from 1 to 1000 amino acids, more preferably from 30 to 1000 amino acids, even more preferably from 50 to 600 amino acids and most preferably from 100 to 300 amino acids.

The size of the amino component may be as little as one amino acid. There is not necessarily any upper limit of the length of the amino component, but it is determined ultimately, if at all, by the specificity of the enzyme used and reaction kinetics considerations, for example the diffusion rate of the amino component. Typical sizes of the amino component are from 1 to 1000 amino acids, more preferably from 30 to 500 amino acids and more preferably from 30 to 250 amino acids. However, it is also within the scope of the present invention that the length of amino component is distinctly greater, especially in those embodiments of the method according to the invention in which a sequential enzyme- or protease-catalyzed peptide fragment ligation is effected or a protein serves as a reactant. The length is then preferably a multiple of the aforementioned length ranges. It is within the scope of the present invention that the amino component is larger, the same size or smaller than the carboxyl component, the criterion used for this purpose generally being the number of amino acids forming the amino component or the carboxyl component.

It is preferred that the carboxyl component has a size of from 1 to 1000 amino acids, preferably from 30 to 500 amino acids, more preferably from 30 to 250 amino acids.

In the method according to the invention, it is preferably provided that the amino components used are N-terminally unprotected peptides, peptide mimetics and proteins.

The reaction is effected preferably in pure ionic liquids, for example 4-methyl-N-butylpyridinium tetrafluoroborate, with only a very small, if any, water content (typically less than 5%).

In a further embodiment of the invention, the proportion of ionic liquids in the reaction medium is 50-100% by volume, preferably 70-100 or 80-100% by volume, more preferably 90-100% by volume, likewise preferably from 95 to 100% by volume, 95-99% by volume or 97-99% by volume.

Mixtures of ionic liquids and organic solvents with and without water content and further additives, for example inorganic salts, likewise constitute reaction media in the context of the invention, and it is unimportant whether it is a solution or suspension. The additives used may in particular be: inorganic salts, buffer components, reducing and oxidizing agents, enzyme activators, modulators and inhibitors, surfactants, lipids, polymers for the covalent or adhesive immobilization of proteins (for example polyethylene glycol, methoxypolyethylene glycol or carboxymethylcellulose) and protein-denaturing agents such as SDS (sodium dodecylsulfate), urea or guanidine hydrochloride.

A crucial advantage of the use of solvent mixtures may lay in the increase or reduction in the solubility of reactants or enzyme, or in the possibility of influencing the enzyme activity and specificity by the number, type and proportion of solvents used in addition to the ionic liquid.

According to the invention, preference is given to using water-miscible organic solvents when they also mix with the ionic liquids. On the other hand, the invention also embraces hydrophobic organic solvents (e.g. hexane or octane) which are water-immiscible, in which case the solvent mixture is present as a biphasic system. Also embraced by the invention is the use of modified ionic liquids having hydrophobic alkyl groups which mix either partly or fully with hydrophobic organic solvents.

According to the invention, preference is given to using ionic liquids in which the cations are alkylimidazolium ions, alkylammonium ions, alkylpyridinium ions and/or alkylphosphonium ions, in which the alkylation is in each case complete, i.e. none of the heteroatoms mentioned is bonded to a hydrogen atom, and are thus quaternized.

Preference is likewise given to the alkyl radicals of the ionic liquids being branched or unbranched and having 1-20 carbon atoms, preferably 1-20 carbon atoms, more preferably 4-6 carbon atoms. Particular preference is given to at least one alkyl radical being methyl, ethyl, propyl or butyl, in particular butyl.

According to the invention, the anions of the ionic liquids which can be used with preference are chloride, bromide, chloroaluminate, nitrate, benzenesulfonate, triflate(trifluoromethanesulfonate), tosylate and/or tetrafluoroborate.

According to the invention, the ionic liquids used are more preferably 1-ethyl-3-methylimidazolium, 1-butyl-3-methylimidazolium and/or 4-methyl-N-butylpyridinium salts, of which the particular tetrafluoroborate is particularly preferred.

The carboxyl components with the preferably enzyme-specific leaving group may be synthesized chemically by condensation of the acyl radical of the carboxyl components with the particular leaving group (or suitable precursors) or else on a polymeric support, for example by using sulfamylbutyrylaminomethyl safety-catch resins (cf. R. Ingenito et al., J. Am. Chem. Soc. 1999, 121, 11369) or oxime resins (cf. V. Cerovsky, F. Bordusa, J. Peptide Res. 2000, 55, 325; V. Cerovsky et al., Chem Bio Chem 2000, 2, 126) with synchronous ester or amide generation and peptide elimination. The releasing nucleophile used is the appropriate alcoholic, phenolic, mercapto- or amino-containing leaving group, or else already prepared N^{α}-unprotected amino acid esters or amides or suitable precursors. Alternatively, the carboxyl component may be synthesized by a genetic engineering route, for example by using intein-mediated polypeptide ester synthesis (M. W. Southworth et al., Biotechniques 1999, 27, 110). The leaving group can be adapted by the selection of the nucleophile in the intein cleavage, or else on completion of ester generation by transesterification in solution.

The synthesis of the peptide fragments used as the amino components is possible routinely in solution or by using conventional Fmoc or Boc synthesis protocol on a polymeric support. Typically, the synthesis on a polymeric support is to be preferred over a more complicated solution synthesis owing to the advantages in the purification of the individual intermediates. Alternatively, the amino components may be obtained from biological material or expressed by genetic engineering methods with subsequent isolation.

The resulting synthesized or labeled products may be separated and purified by customary methods of peptide and protein chemistry. Any protecting groups present may be removed by methods known in the prior art.

In a preferred embodiment of the invention, a compound prepared by the steps a) to c) and optionally d) may be used as the amino component and another compound prepared by steps a) to c) and optionally d) as the carboxyl component in order to build up larger polypeptides or proteins from defined constituents which, depending on the method, may also have label or reporter groups.

In contrast to other potentially usable biocatalytic methods, the inventive use of ionic liquids or mixtures thereof in combination with the use of enzymes and in particular proteases and peptidases achieves a high synthesis rate, flexibility, synthesis efficiency and simplicity in the handling.

The present invention is illustrated with reference to the drawings and examples, from which further features, embodiments and advantages of the invention are evident.

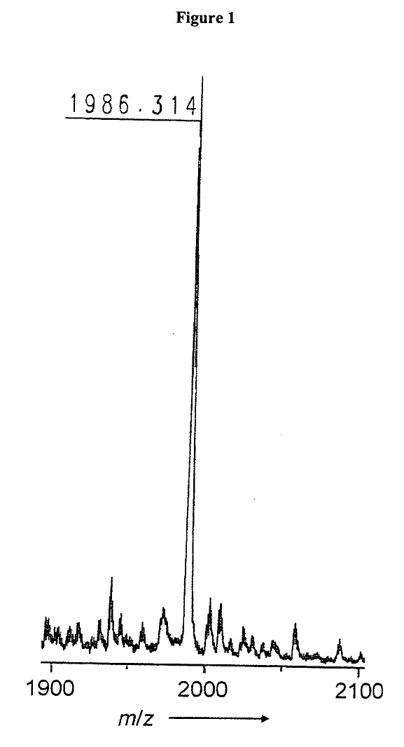

FIG. 1 shows a selected MALDI-ToF mass spectrum of the biotinylated peptide prepared by application of the method according to the invention and as described in example 4.

### EXAMPLES

### Example 1

Influence of the proportion of the ionic liquid 4-methyl-N-butylpyridinium tetrafluoroborate on the trypsin-catalyzed synthesis of di- and tripeptides (Bz, benzoyl; OGp, 4-guanidinophenyl ester).

1 ml of reaction solution which contains the mixtures of 4-methyl-N-butylpyridinium tetrafluoroborate specified in tab. 1 and also 0.1 M Hepes buffer pH 8.0, containing 0.1 M NaCl and 0.01 M CaCl₂, 1.5% (v/v) 4-methylmorpholine, 2 mM Bz-Phe-OGp, 20 mM amino component and 10 μM trypsin, is stirred at 25° C. After 30-120 min, the reaction solution is brought to pH 2 using 1% trifluoroacetic acid in methanol/water (1:1, v/v). The yields of di- and tripeptide were determined at HPLC analysis and are listed in the following table 1.

Bz-Phe-OGp was synthesized analogously to the synthesis protocol of M. Thormann et al., Biochemistry 1999, 38, 6056. The amino components used are commercially available products and are specified in table 1. Trypsin was obtained from Fluka (Switzerland).

TABLE 1

| Proportion of | | | | | |
|---|---|---|---|---|---|
| ionic liquid | | | | | |
| (v/v) | H-Leu-NH₂ | H-Gly-NH₂ | H-Met-NH₂ | H-Ser-NH₂ | H-Ala-Met-OH |
| 0 | 75.2 | 45.2 | 68.0 | 52.1 | 51.5 |
| 20 | 77.4 | 49.1 | 69.3 | 54.5 | 53.9 |
| 40 | 79.7 | 54.1 | 72.9 | 58.4 | 57.6 |
| 60 | 85.8 | 62.4 | 77.0 | 68.9 | 68.5 |
| 80 | 87.3 | 64.7 | 79.0 | 70.4 | 71.4 |

### Example 2

Influence of the type and the amount of additional organic solvents on the trypsin-catalyzed synthesis of Bz-Phe-Leu-NH₂ starting from Bz-Phe-OGp and H-Leu-NH₂ in the ionic liquid 4-methyl-N-butylpyridinium tetrafluoroborate (Bz, benzoyl; OGp, 4-guanidinophenyl ester; MeOH, methanol; DMSO, dimethyl sulfoxide; DMF, dimethylformamide).

1 ml of reaction solution which contains 4-methyl-N-butylpyridinium tetrafluoroborate, 5% water and the specified proportions of additional organic solvent, 1.5% (v/v) 4-methylmorpholine, 2 mM Bz-Phe-OGp, 20 mM amino components and 20, 40, 80, 200 μM trypsin (with increasing proportion of additional organic solvent) is stirred at 25° C. After 30-120 min, the reaction solution is brought to pH 2 using 1% trifluoroacetic acid in methanol/water (1:1, v/v). The yields were determined by HPLC analysis and are listed in the following table 2.

TABLE 2

| Organic | Product yield (%)/ionic liquid: organic solvent ratio | | | |
|---|---|---|---|---|
| Solvent | 50:50 (v/v) | 60:40 (v/v) | 70:30 (v/v) | 80:20 (v/v) |
| MeOH | 93.2 | 93.7 | 93.8 | 94.7 |
| DMSO | 91.9 | 92.4 | 92.8 | 94.8 |
| DMF | 91.7 | 92.0 | 92.7 | 93.7 |

### Example 3

Trypsin-catalyzed synthesis of polypeptides with enzyme-specific cleavage sites starting from Bz-Phe-OGp and polypeptides of different length and sequence in the ionic liquid 4-methyl-N-butylpyridinium tetrafluoroborate.

The individual enzyme-specific amino acid residues are each emphasized by bold type (Bz, benzoyl; OGp, 4-guanidinophenyl ester).

1 ml of reaction solution which contains 4-methyl-N-butylpyridinium tetrafluoroborate, 5% water, 1.5% (v/v) 4-methylmorpholine, 2 mM Bz-Phe-OGp, mM amino components and 10 μM trypsin is stirred at 25° C. For solubility reasons, the reactions were carried out with methanol as an additional organic solvent, and both a proportion of 20% and 50% (v/v) of methanol was used. After 30-120 min, the reaction solution is brought to pH 2 using 1% trifluoroacetic acid in methanol/water (1:1, v/v). The polypeptide products identified by means of MALDI-ToF after they had been isolated from the reaction solution are listed in table 3 with particular calculated and found molecular masses.

TABLE 3

| | Calculated | Found |
|---|---|---|
| | mass | mass |
| Synthesis product | [g/mol]* | [g/mol] |
| Bz-FAARAG | 695.34 | 696.3 (+H⁺) |
| Bz-FRIVDARLEQVKAAGAY | 2010.07 | 2025.09 (+Na⁺) |
| Bz-FRIVDAVLEQVKAAGAY | 1953.04 | 1954.31 (+H⁺) |
| Bz-FKVVFSAPV LEPTGPLHTQ | 3673.98 | 3690.50 (+Na⁺) |
| FGYHIIKVLY RN | | |

*Specification of the monoisotopic masses

### Example 4

Trypsin-catalyzed N-terminal introduction of the biotin label group into polypeptides having enzyme-specific cleavage sites starting from biotinyl-OGp and polypeptides of different length and sequence in the ionic liquid 4-methyl-N-butylpyridinium tetrafluoroborate.

The individual enzyme-specific amino acid residues are each emphasized by bold type (Bz, benzoyl; OGp, 4-guanidinophenyl ester).

The reaction conditions correspond to those of example 3. The only change was in the concentration of carboxyl component (biotinyl-OGp: 4 mM) and amino component (each peptide: 2 mM). The polypeptide products identified by means of MALDI-ToF after they have been isolated from the reaction solution are listed in table 4 with the particular calculated and found molecular masses. The selectivity of the enzymatic biotinylation reactions was investigated by using N-terminally acetylated peptide analogs. The absence of product formation in these cases was assessed as confirmation of an exclusive N-terminal biotinylation.

TABLE 4

| | Calculated | Found |
|---|---|---|
| | mass | mass |
| Synthesis product | [g/mol]* | [g/mol] |
| Biotinyl-RIVDARLEQVKAAGAY | 1985.05 | 1986.31 (+H⁺) |
| Biotinyl-KVVFSAPV LEPTGPLHTQ | 3648.96 | 3649.67 (+Na⁺) |
| FGYHIIKVLY RN | | |

*Specification of the monoisotopic masses

The resulting MALDI-ToF mass spectrum of the biotinyl-RIVDARLEQVKAAGAY synthesis product is shown by way of example in FIG. 1. The found molar mass of 1986.31 corresponds to the (M+H⁺) signal of the monoisotopic molar mass of the peptide product calculated at 1985.05.

### Example 5

Chymotrypsin-catalyzed selective introduction of the biotin label group into lysozyme from chicken egg white in the ionic liquid 4-methyl-N-butylpyridinium tetrafluoroborate.

Chymotrypsin has a relatively brad substrate specificity, although the enzyme cleaves preferentially after aromatic amino acid residues. The individual aromatic amino acid residues occurring in the lysozyme are each emphasized by bold type (Hepes, N-[2-hydroxyethyl]piperazine-N′-[2-ethanesulfonic acid); OGp, 4-guanidinophenyl ester).

Primary sequence of lysozyme from chicken egg white:

KVFGRCELAA AMKRHGLDNY RGYSLGNWVC QATNRNTDGS
TDYGILQINS RWWCNDGRTP GSRNLCNIPC SALLSSDITA
SVNCAKKIVS DGNGMNAWVA QAWIRGCRL

1 ml of reaction solution which contains 4-methyl-N-butylpyridinium tetrafluoroborate, 20% Hes buffer (0.05 M, pH 8.0), 2 mM biotinyl-OGp, 0.5 mM lysozyme and 10 μM chymotrypsin is stirred at 25° C. After 120 min, the reaction solution is brought to pH 2 using 1% trifluoroacetic acid in methanol/water (1:1 v/v). The molar mass, found by means of MALDI-ToF after product isolation, of 14589.06 of the synthesis product corresponds to the (M+H⁺) signal of the theoretically calculated monoisotopic molar mass of monobiotinylated lysozyme.

The features, disclosed in the above description, the claims and the drawings, of the invention may be used either individually or in any combinations for the realization of the invention in different embodiments.

What is claimed is:

1. A method for the synthesis of peptides, peptide mimetics and/or proteins and/or for the selective N-terminal modification of peptides, peptide mimetics and/or proteins, with the steps of:
a) providing an amino component, said amino component having at least one amino acid,
b) providing a carboxyl component, said carboxyl component having a leaving group on the carboxyl group, and said carboxyl component being a compound having at least one amino acid or a compound having at least one label or reporter group,
c) reacting said amino component and said carboxyl component in a reaction medium which has one or more ionic liquids, in the presence of a protease, peptidase and/or hydrolase, to form a peptide bond between the amino component and the carboxyl component with elimination of the leaving group.

2. The method as claimed in claim 1, further comprising the step of:
d) enriching and/or isolating the resulting peptide, peptide mimetic and/or protein by methods known per se.

3. The method as claimed in claim 1 or 2,
characterized in that
the amino component is a polypeptide or protein.

4. The method as claimed in one or more of the preceding claims,
characterized in that
the amino component has a size of from 1 to 1000 amino acids, preferably from 30 to 500 amino acids, more preferably from 30 to 250 amino acids.

5. The method as claimed in one or more of the preceding claims,
characterized in that
the carboxyl end of the amino component is present protected or unprotected in nonactivated form.

6. The method as claimed in one or more of the preceding claims,
characterized in that
the N^{α}-amino function of the amino component is present unprotected, and this N^{α}-amino function reacts with the carboxyl function of the carboxyl component.

7. The method as claimed in one or more of the preceding claims,
characterized in that
the carboxyl component is a polypeptide or protein.

8. The method as claimed in one or more of the preceding claims,
characterized in that
the carboxyl component has a size of from 1 to 1000 amino acids, preferably from 30 to 500 amino acids, more preferably from 30 to 250 amino acids.

9. The method as claimed in one or more of the preceding claims,
characterized in that
the carboxyl group of the carboxyl component forms a carboxylic ester or a carboxamide with the leaving group.

10. The method as claimed in one or more of the preceding claims,
characterized in that
the leaving group of the carboxyl component is selected from the group consisting of unsubstituted and substituted —O-alkyl-, —O-aryl-, —S-alkyl-, —S-aryl radicals, —NH-alkyl-, —NH-aryl-, —N,N-dialkyl-, —N,N-diaryl- and —N-aryl-N-alkyl-radicals.

11. The method as claimed in claim 10,
characterized in that
the leaving group of the carboxyl component is substituted by one or more carboxylic acid radicals, sulfonic acid radicals or sulfonates.

12. The method as claimed in one or more of the preceding claims,
characterized in that
the leaving group is a 4-guanidinophenyl, 4-amidinophenyl, 4-guanidinophenylthio or 4-amidinophenylthio radical, or a compound structurally homologous thereto.

13. The method as claimed in one or more of the preceding claims,
characterized in that
the leaving group is adjusted to the specificity of the protease, peptidase and/or hydrolase used.

14. The method as claimed in one or more of the preceding claims,
characterized in that
the carboxyl component containing the leaving group has the following structure:
Y—(Xaa)ₙ-R
where Y=an N-terminal protecting group or is H,
Xaa=any α-amino acid, β-amino acid or a derivative thereof, or is a label or reporter group,
R is a leaving group, in particular a leaving group which is selected from the group consisting of unsubstituted and substituted —O-alkyl, —O-aryl-, —S-alkyl-, —S-aryl-radicals, preferably 4-guanidinophenyl, 4-amidinophenyl, 4-guanidinophenylthio-, 4-amidinophenylthio radicals, each of which may be substituted by sulfonic acid groups or sulfonates, and also structural homologs thereof,
n is an integer of from 1 to 1000, preferably from 30 to 500, more preferably from 30 to 250.

15. The method as claimed in one of more of the preceding claims,
characterized in that
the carboxyl component is a label or reporter group selected from fluorescent labels containing carboxyl groups, such as fluorescein, rhodamine, tetramethylrhodamine, 2-aminobenzoic acid, isotopic labels containing carboxyl groups, such as ¹³C—, ¹⁵N— and ¹⁷O-containing amino acids or peptide fragments; spin labels containing carboxyl groups, such as nitroxide label-containing amino acid and fatty acid derivatives; biotin; crosslinking agents containing carboxyl groups, such as diazoacetate, diazopyruvate, p-nitrophenyl-3-diazopyruvate; 2-(1,2-dithiolan-3-yl)acetate; N,N′-1,2-phenylenedimaleimide; N,N′-1,4-phenylenedimaleimide.

16. The method as claimed in one or more of the preceding claims,
characterized in that
the steps a) to c) and optionally d) are carried out twice or more, in order to prepare sequentially a polypeptide or protein which may have a label or reporter group.

17. The method as claimed in one or more of the preceding claims,
characterized in that
a compound prepared according to steps a) to c) and optionally d) is used as the amino component and another compound prepared according to steps a) to c) and optionally d) is used as the carboxyl component.

18. The method as claimed in one or more of the preceding claims,
characterized in that
the reaction medium has exclusively one or more ionic liquids.

19. The method as claimed in one or more of claims 1 to 17,
characterized in that
the reaction medium comprises one or more ionic liquids and also water, and/or an organic solvent and optionally customary additives.

20. The method as claimed in one or more of the preceding claims,
characterized in that
the proportion of ionic liquids in the reaction medium is 50-100% by volume, preferably 70-100 or 80-100% by volume, more preferably 90-100% by volume, likewise preferably from 95 to 100% by volume or 95-99% by volume.

21. The method as claimed in one or more of the preceding claims,
characterized in that
the cations of the ionic liquids used are quaternized alkylimidazolium ions, quaternized alkylammonium ions, quaternized alkylpyridinium ions and/or quaternized alkylphosphonium ions.

22. The method as claimed in claim 21,
characterized in that
the alkyl radicals of the ionic liquids are branched or unbranched and have 1-20 carbon atoms, preferably 2-10 carbon atoms, more preferably 4-6 carbon atoms.

23. The method as claimed in one or more of the preceding claims,
characterized in that
the ionic liquids used are 1-ethyl-3-methylimidazolium, 1-butyl-3-methylimidazolium and/or 4-methyl-N-butylpyridinium salts.

24. The method as claimed in one or more of the preceding claims,
characterized in that
the anions of the ionic liquids used are chloride, bromide, chloroaluminate, nitrate, benzenesulfonate, triflate(trifluoromethanesulfonate), tosylate and/or tetrafluoroborate.

25. The method as claimed in one or more of the preceding claims,
characterized in that
the protease used is a cysteine protease or serine protease.

26. The use of ionic liquids as an exclusive solvent or in combination with water and/or organic solvents for the synthesis and/or N-terminal modification of peptides, peptide mimetics and/or proteins.

27. The use of a protease, peptidase and/or hydrolase for the synthesis and/or N-terminal modification of peptides, peptide mimetics and proteins, said peptide, peptide mimetic and protein or N-terminally labeled species thereof being prepared by ligation of an amino component and a carboxyl component, and said carboxyl component having a leaving group.
